(19) United States
(12) Patent Application Publication
(10) Pub. No.: US 2016/0220174 A1
(43) Pub. Date: Aug. 4, 2016

(54) Body-Sensing Tank Top with Biofeedback System for Patients with Scoliosis

(71) Applicant: THE HONG KONG POLYTECHNIC UNIVERSITY, Hong Kong (HK)

(72) Inventors: Yiu-Wan YIP, Hong Kong (HK); Xiao-Chuan YU, Hong Kong (HK); Toong-Shoon Alvin CHAN, Hong Kong (HK); Mei-Chun CHEUNG, Hong Kong (HK); Kit Lun YICK, Hong Kong (HK); Sun Pui NG, Hong Kong (HK); Chi Yung TSE, Hong Kong (HK); Garcia Hin Chun KWOK, Hong Kong (HK)

(21) Appl. No.: 14/613,337

(22) Filed: Feb. 3, 2015

(51) Int. Cl. A61B 5/00; A61B 5/11; A61B 5/01; A61B 5/0488; A61B 5/05

(52) U.S. Cl. CPC A61B 5/4561; A61B 5/0488; A61B 5/05; A61B 5/01; A61B 5/11; A61B 5/486; A61B 5/6804; A61B 5/7264; A61B 5/0022

(57) ABSTRACT

A garment, in a form of tank top, for monitoring patient-related signals of a patient having scoliosis and thereby enabling the patient to obtain a personalized biofeedback is provided. The garment are integrated with plural sensors, a sensor interface and a smart control unit (SCU), allowing the patient-related signals to be non-intrusively measured by the sensors while maintaining comfort to the patient when the patient wears the garment. The SCU is communicable with the sensors via the sensor interface and aggregates the patient-related signals. A computing server outside the garment receives the aggregated patient-related signals from the SCU via a user access device such as a smartphone, and processes the aggregated patient-related signals to generate the personalized biofeedback, which is then forwarded to the user access device for presentation to the patient. Machine learning algorithms are used to process the patient-related signals in generating the biofeedback.

### FIELD OF THE INVENTION

The present invention relates to a garment for monitoring patient-related signals of a patient having scoliosis and thereby enabling the patient to obtain a personalized biofeedback based on the patient-related signals and generated from a computing server.

### BACKGROUND

Adolescent idiopathic scoliosis (AIS) is a multi-factorial, three-dimensional deformity of the spine and trunk which can appear and sometimes progress during any of the rapid periods of growth in apparently healthy children. For non-surgical and non-medical interventions, conventional orthotic interventions apply passive forces to the human body with orthosis to support the trunk alignment and control the deformities of the spine. However, the use of these external supports is limited by factors such as poor appearance, bulkiness, physical constraint, and muscle atrophy that could lead to low acceptance and compliance. Back muscle strengthening exercises attempt to strengthen the back muscles to maintain the trunk in an upright position with active muscular forces. However, patient compliance with the prescribed intervention exercises present a challenge, especially patients who are not self-motivated may not continue with the prescribed exercise programs.

There are a few existing works focusing on adopting sensor-based technology in treating idiopathic scoliosis. In WO2013110835A1, a programmable subcutaneous or submuscular device is proposed to collect/record electromyographic signals and stimulate that part of the deep paraspinal muscles that is affected by the pathology. The muscle stimulation is controlled by control logic that comprises a feedback-loop algorithm for adjustment of the stimulation on the basis of the results obtained from the sensors. There are many drawbacks regarding to this design. First, it is intrusive. The submuscular module requires proper procedure to be implanted into human body. This requirement largely affects comfort and compliance of the system, and even causes side effects such as infection. Second, it relies on a naive feedback loop. The feedback loop is implemented locally using predefined control logic. This imposes difficulty in modifying the feedback algorithm once the device is setup. More importantly, it has intrinsic inability to support adaptation of the feedback logic based either on the historical information such as patient's progress, or on external information such as doctor/specialists' opinion. Third, wired connection is adopted on the body area. Compared to wireless setup, wired design is less flexible, and less comfortable for the patient. Fourth, only electromyography is considered. It lacks the consideration of other important factors like patient's motion, posture, etc.

In U.S. Pat. No. 5,082,002A, a system and method for the operant conditioning of subjects using biofeedback is proposed. The design provides means to measure a variable condition, such as posture, which is controllable by the subject. The apparatus sets criteria, which, if not met, may result in a negative reinforcement, such as unpleasant audio tone or, if the criteria are met, will reward the subject. The criterion is automatically adjusted, upwards or downwards, in accordance with the subject's history of reaching, or not reaching, the criteria. Even though this design considered the aspect of adaptation, the adaptation method it used is very primitive—it is achieved by adjusting criteria upwards or downwards. In applications, however, the criteria are hard to set because multiple metrics (resulting to multitude of criteria) should be considered, let alone each criterion should vary from patient to patient. Hence, simply using criterion-based detection in this scenario is not sufficient. Another drawback of this design is that it proposed a tension-based sensor to detect the posture of the patients. Compared to a modern motion sensor, which utilizes accelerometer and gyroscope, the tension-based sensor lacks precision, flexibility, and is prone to error (due to the strict placement requirement).

Regarding the posture control, which is a major consideration for AIS treatment, the state-of-the art posture correction techniques usually consist of three abstract components: (1) feedback loop; (2) posture sensors; and (3) feedback means. Existing works on posture control are summarized in accordance with each respective component as follows.

Most of the designs, e.g., in WO2013110835A1, US20130108995A1, U.S. Pat. No. 8,157,752B2, U.S. Pat. No. 7,850,574B2, US20090054814A1, WO2006062423A1, U.S. Pat. No. 6,673,027B2 and U.S. Pat. No. 6,579,248B1, adopted a feedback loop with predefined (normally hardcoded) control logic, which we name as a naive feedback loop. The control logic or switch circuit is normally established based on one or a few preset criteria. The feedback means (such as an audio alert) is triggered when given criterion are reached. The whole control flow is normally implemented in hardware (using a switch circuit) as in U.S. Pat. No. 5,158,089A, U.S. Pat. No. 5,082,002A, U.S. Pat. No. 4,914,423A, U.S. Pat. No. 4,750,480A, U.S. Pat. No. 4,730,625A, U.S. Pat. No. 4,007,733A and U.S. Pat. No. 5,168,264A, or is hardcoded in software control logic on microcontrollers as in US20130108995A1, WO2013110835A1 and U.S. Pat. No. 8,157,752B2. As mentioned before, the naive feedback mechanism imposes difficulty in modifying the feedback algorithm once the device is set-up. More importantly, it has intrinsic inability to support adaptable feedback logic.

As for posture sensors, inclination (also pendulum) (U.S. Pat. No. 5,168,264A, U.S. Pat. No. 5,158,089A, US20090054814A1), tension (U.S. Pat. No. 4,007,733A, U.S. Pat. No. 4,914,423A, U.S. Pat. No. 5,082,002A, U.S. Pat. No. 5,728,027A, U.S. Pat. No. 6,384,729B1, U.S. Pat. No. 6,579,248B1, WO2006062423A1, US20080319364A1 and U.S. Pat. No. 8,083,693B1), flowable substance (U.S. Pat. No. 7,980,141B2), hinge (U.S. Pat. No. 6,673,027B2), distance between body and sensor (U.S. Pat. No. 8,157,752B2), have been used as sensory means for posture detection in earlier designs. While effectiveness of these methods is largely dependent on the application area and the positioning of sensory devices, the accuracy of a reading cannot always be maintained on an acceptable confidence level. Therefore, to be able to adopt these methods, a more sophisticated design is applied, leading to a poor appearance, bulkiness, and one or more physical constraints in a final design, all of which would in turn affect effectiveness and compliance of the devices. There are some designs embracing modern motion detection approaches that use accelerometers (or combined with gyroscopes), as in US20110063114A and US20130108995A1. Using such type of sensors can acquire more reliable data inputs and enable more flexible designs. However, providing an efficient detection mechanism that fully utilizes such sensor readings is still a challenging issue. Especially in the area of posture correction, it is impossible to define an absolutely correct posture out of the measurement provided by the sensors. In this case, the naive feedback algorithm with a threshold-based detection algorithm that most existing works have proposed would not suffice.

Very limited feedback means have been adopted in existing techniques. Specifically, only sound and vibration are utilized in a form of alert (a.k.a. notification). However, as mobile devices such as smartphones and tablets have become increasingly pervasive, more user-friendly feedback means can be advantageously provided through those devices. To be more specific, feedback should not only limited to the form of alert, but integrated into existing mobile platforms, utilizing frameworks such as HealthKit on iOS or Google Fit on Android, as well as interfacing with established social media platforms (e.g. Facebook and Twitter) and healthcare platforms (e.g. Mayoclinic: www.mayoclinic.org).

There is a need in the art to have an improved device over existing ones for treating AIS.

### SUMMARY OF THE INVENTION

The present invention provides a garment for monitoring patient-related signals of a patient having scoliosis and thereby enabling the patient to obtain a personalized biofeedback based on the patient-related signals. The garment comprises plural sensors, a sensor interface and a smart control unit (SCU), all integrated in the garment, allowing the patient-related signals to be non-intrusively measured by the sensors while maintaining comfort to the patient when the patient wears the garment. The SCU is communicable with the sensors via the sensor interface and is configured to aggregate the patient-related signals measured by the sensors. In addition, the SCU is configured to be communicable with a computing server outside the garment via a user access device. The computing server is configured to process the aggregated patient-related signals sent from the SCU to generate the personalized biofeedback, and configured to forward the personalized feedback to the user access device for presentation to the patient. In particular, the garment is configured to electrically power the sensors, the sensor interface and the SCU but neither the user access device nor the computing server. It follows that the personalized biofeedback is obtainable by the patient without a need for the garment to spend electrical power to process the patient-related signals in generating the personalized biofeedback.

Preferably, the garment is fabricated as a tank top.

The present invention also provides a system for monitoring patient-related signals of a patient having scoliosis and for providing a personalized biofeedback to the patient based on the patient-related signals. The system comprises the garment as disclosed, the user access device and the computing server.

Other aspects of the present invention are disclosed as illustrated by the embodiments hereinafter.

### BRIEF DESCRIPTION OF THE DRAWINGS

FIG. 1 is a conceptual diagram depicting an operating scheme of a system for illustrating the present invention.

FIG. 2 depicts an architecture of the system of FIG. 1.

FIG. 3 is, in accordance with an exemplary embodiment of the present invention, a schematic diagram of a garment and a system for monitoring patient-related signals of a patient having scoliosis and for providing a personalized biofeedback to the patient based on the patient-related signals.

FIG. 4 depicts four example designs of the garment, each of the designs being a tank top.

### DETAILED DESCRIPTION

The following definitions are used herein in the specification and the appended claims. “A cloud” is construed and interpreted in the sense of cloud computing or, synonymously, distributed computing over a network unless otherwise specified. “A server” or “a computing server” is interpreted in the sense of computing. The one or more storages may be, for example, hard disks or solid-state disk drives. A server is generally equipped with one or more processors for executing program instructions, and one or more storages for storing data. A server may be a standalone computing server, or a distributed server in the cloud.

Regarding all the issues of existing methods and systems for treating AIS as mentioned above, the present invention addresses these issues by aiming to develop an innovative body-sensing garment, in a preferred form of a tank top, equipped with a biofeedback system for adolescents with early scoliosis. The system provides muscle re-education at specific areas, including the upper trapezius, thoracic and lumbar regions, so as to strengthen muscle strength and train the individual into adopting the desired posture during sitting and standing, which is very useful for the prevention and/or controlling of the curve progression of spinal deformities.

Particularly, the present invention provides a compact, non-intrusive wearable computing platform to provide real-time data surveillance, notification, and motivational program for the patients through their daily activities and exercises. Via long-term and continuous use, the platform can deliver analysis and intervention techniques that used to be only available inside institute/laboratory environment. It is also believed that the sensor-based biofeedback device can motivate patients to play an active role, thus improving their control and coordination of movement and daily posture more efficiently. The data acquired from the device can be further provided to doctors or specialists in a timely manner.

As is mentioned above, the naive feedback algorithm with threshold based detection that most of the existing works employ does not suffice. The present invention mitigates this shortcoming by involving a more advanced data processing method such as machine learning algorithm into the feedback loop, to provide adaptive, personalized feedback. Machine intelligence is used to combine and process information on the patient's behavior pattern, expert knowledge (doctor's diagnostic opinion, instructions, etc.), and predefined profiles created by the patient and his doctor. As a result, more accurate, dynamic and personalized feedback can be provided to the patient. Besides the diagnostic surveillance and posture correction in the patient's daily activities, the platform as disclosed in the present invention is also utilizable to progressively facilitate customized muscle training sessions to the patient with scoliosis so as to restore a balance in muscle activity and reduction in the displacement of both sides of the spine.

In short, the present invention is concerned with a sensor-based wearable biofeedback system, in which real-time data about a patient's posture, motion, as well as other vital signal's such as body temperature, muscle activities are recorded, stored in both local and cloud-based databases and analyzed by machine-learning algorithms that combine and process information on patient's behavior pattern, expert knowledge (e.g. doctor's instruction), and predefined profiles (created by the patient and his doctor), so as to provide dynamic, personalized feedback means to the user.

Exemplarily, the present invention is illustrated according to an exemplary design of the sensor-based wearable biofeedback system disclosed in Section A below. After the design of this system and the advantages thereof are elaborated, the present invention is detailed in Section B.

### A. An Exemplary Design of the Sensor-Based Wearable Biofeedback System

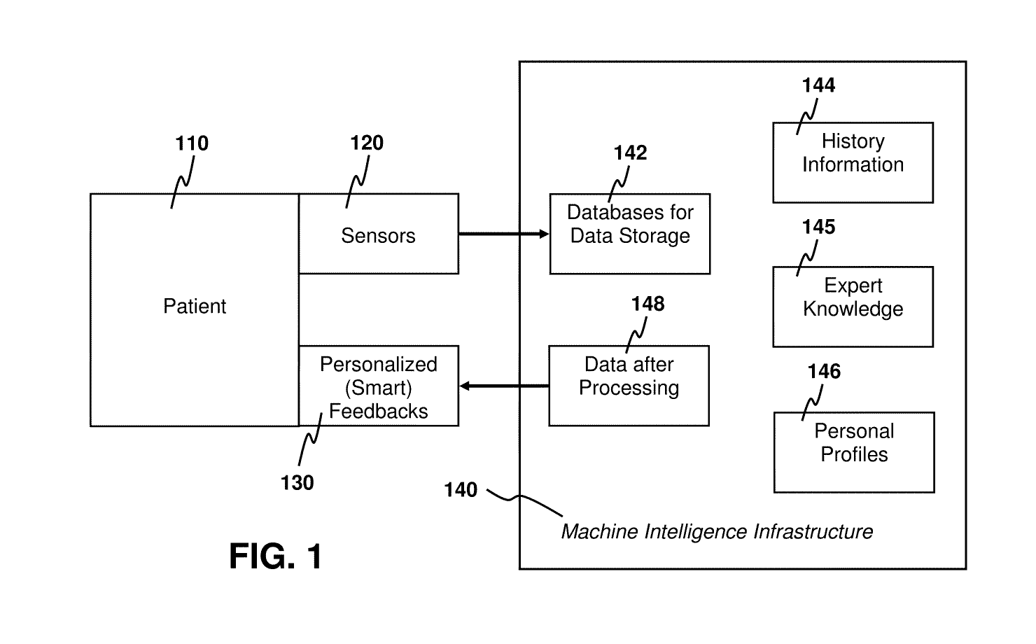

FIG. 1 is a conceptual diagram depicting the system's operating scheme. Real-time data about a posture, a motion, etc. of a patient 110, as well as other vital signal thereof such as muscle activities are recorded by sensors 120, stored in databases 142 (local and/or cloud-based) and analyzed by machine-learning algorithms provided by a novel machine intelligence infrastructure 140. Utilizing the computation power of cloud computing infrastructures, the machine intelligence infrastructure 140 combines and processes information on the patient 110's history information 144 (e.g., the patient 110's behavior patterns), expert knowledge 145 (e.g., a doctor's instruction), and predefined profiles 146 created by the patient 110 and the doctor thereof to yield processed data 148, and consequently sends the processed data 148 as dynamic, personalized feedbacks 130 (Smart Feedback) that are deeply integrated to existing mobile platforms, social network service platform and healthcare service platforms.

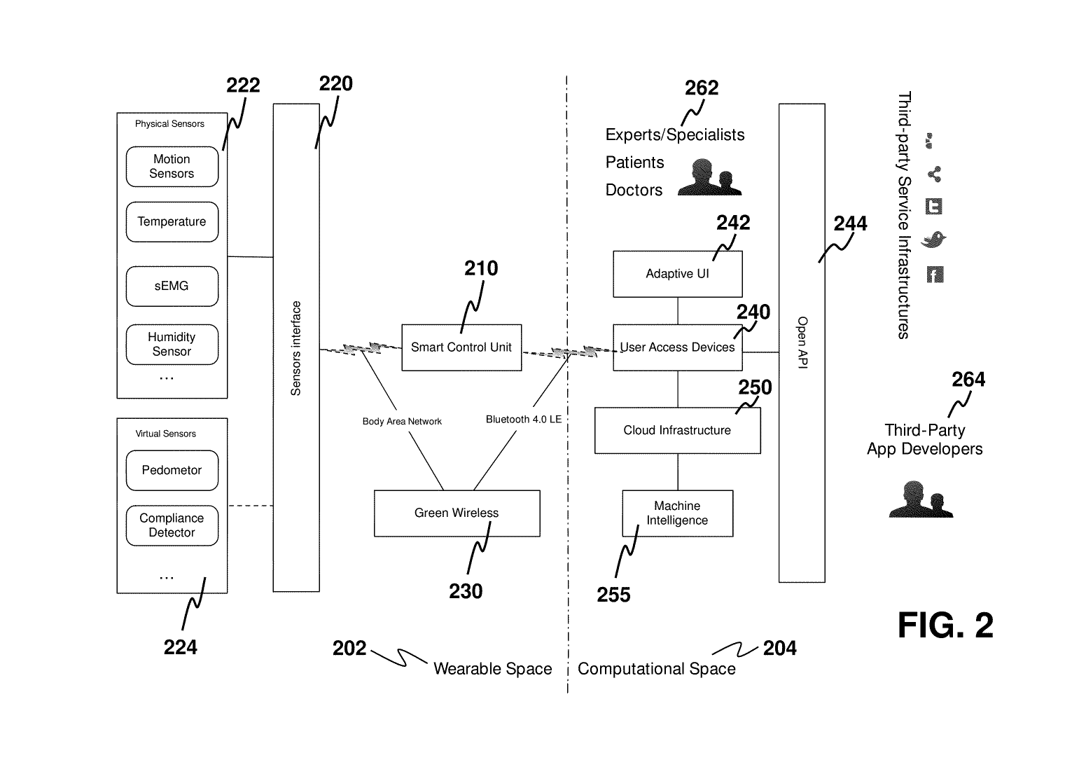

FIG. 2 depicts an architecture of the system. The system comprises components that reside in a wearable space 202, i.e. devices embedded in a garment, and components that reside in a computational space 204, i.e. devices and facilities that are used for user access and computation. Description for each component is provided as follows.

### A.1. Sensors (Physical Sensors 222 and Virtual Sensors 224)

Sensors used in the system can be physical or virtual. A physical sensor 222 used in the design contains, but not limited to: a 3-axis accelerometer, a 3-axis gyroscope, a magnetometer (a compass), a surface electromyography (sEMG) sensor, a temperature sensor, and a humidity (moisture) sensor. A virtual sensor 224 is an abstract entity that combines two or more component sensors through sensor fusion algorithms. For instance, a compliance detector is a combination of one or multiple motion sensors and temperature sensors, and is used for detection of user compliance of individual devices.

### A.2. Smart Control Unit 210

A smart control unit (SCU) 210 is used to aggregate the sensor data measured by the sensors 222, 224. After a preliminary process (mainly data encapsulation and format conversion), the SCU 210 sends the results to an user access device 240, to be introduced soon, in the computational space 204 for processing the measured sensor data. The major task of the SCU 210 is to offer a uniform and device-independent data access interface for the user access device 240. Caching techniques may also be adopted to guarantee smooth data transmission between the user access device 240 and the SCU 210. An advantage of the design is that most of the control logics are shifted to the computational space 204. Hence, the architecture of the SCU 210 can be extremely concise (minimalistic): it mainly includes a microcontroller component (with a rechargeable battery) and communication modules. This simplistic design allows for better energy efficiency, as well as compactness in design. This advantage is crucially important as all devices in the wearable space 202 have to be embedded in the garment, so that these devices have to be small, and preferably they can keep operating long enough per battery charge for normal daily usage. The SCU 210 is programmable; the code executed in the SCU 210 needs to guarantee compatibility among different models. In addition, an upgrade feature may be provided through the user access device 240 to guarantee forward compatibility when major changes on any access protocol has been made.

In the design, the communication between the SCU 210 and other components is achieved by communication modules including: i²c, and serial (COM) communication for wired connection; and WBAN and Bluetooth 4.0 LE for wireless communication.

### A.3. Sensor Interface 220

To handle various types of sensors (the physical sensors 222 and the virtual sensors 224), a component—a sensor interface 220—is designed to bridge the sensors 222, 224 to the SCU 210. The sensor interface 220 supports two major functions.
1. It provides common communication protocol support, including wired communication and wireless communication, to connect a variety of sensors 222, 224 to the SCU 210. To be more specific, in the sensor interface 220, wired communication includes: i²c, and serial communication. Wireless communication mainly uses WBAN (IEEE802.15).
2. It provides transformation and encapsulation of data formats supported by various types of sensors 222, 224. That is, it translates the data output from any of different sensors into a unified form that is understandable by the SCU 210.

### A.4. Green Wireless Communication Protocols 230

One major advantage of the design is the adoption of state-of-the-art (green) wireless low energy protocols 230, namely, Bluetooth 4.0 LE, and Wireless Body Area Network (WBAN). Specifically, Bluetooth is mainly used for connection between the user access device 240 and the SCU 210 while the WBAN is mainly used for connections between each of the sensors 222, 224 and the SCU 210. The use of the green wireless protocols 230 can dramatically enhance flexibility in design, and consequently, the comfort to the user (i.e. the patient), while keeping energy consumption marginal.

### A.5. User Access Device 240

The sensor data collected by the SCU 210 are forwarded to a user access device 240 for further processing and analysis. Typically, the user access device 240 can be a smartphone (iPhone, Android Phone, etc.), or a tablet (iPad, Android Tablets, etc.). The PC/MAC may also be partially supported through a Web Interface (only for access to stored user data). A software framework is used for the mobile platform (specifically, for IOS and Android), to provide required libraries and interfaces for the corresponding platform. This framework is the foundation of higher-level functions such as adaptive UI 242. It also offers an interface with an underlying cloud infrastructure, and handles communications from the SCU 210. Various applications can be built utilizing this framework. Deep OS integration is also supported, utilizing cutting-edge tools and infrastructure supports provided by each of the mobile platforms, including CloudKit (IOS), HealthKit (IOS), Google Fit (Android), etc.

### A.6. Cloud Infrastructure 250

A cloud infrastructure 250 is used for data storage and computation-heavy tasks. As predominant mobile platforms, i.e. IOS and Android, already have limited cloud infrastructure supports, built-in features in these platforms are utilized to store platform invariant data such as user profiles. This arrangement provides profile synchronization and application data migration capabilities. Due to the limitation of the above-mentioned infrastructure support and the consideration of platform independence, the data analysis process, such as machine learning algorithm, and other computation-intensive tasks are implemented on an independent cloud infrastructure. Existing cloud-based machine learning utilities can be used for this purpose, such as Google Prediction API, and Microsoft Azure Machine Learning.

### A.7. Machine Intelligence 255

Machine learning algorithms are implemented on the cloud-computing infrastructure 250 to offer machine intelligence 255. A series of data representation, evaluation, and optimization functional modules are constructed based on the data acquired from the sensors 222, 224 on the patient, as well as instructions/knowledge acquired from doctors/experts 262, to provide a knowledge-driven information process and feedback control logic. By using machine intelligence 255, it is feasible to break the barrier due to using conventional naive feedback and threshold-based detection algorithms, thus providing more accurate, dynamic and personalized feedback means for the patient.

The cloud computing approach enables parallelization in a computation process, dramatically speeding up the data analysis and processing required by the machine learning algorithms. It is crucial for the design, as it allows sophisticated pattern recognition and intelligent decisions to be made in nearly real time.

Through machine learning, multitude of functions can be delivered to users. Some examples are given as follows.
1. Via unsupervised learning, certain patterns existed in the data acquired from the sensors 222, 224 can be discovered. This information can be used to identify the user's behavior (e.g., activities that the user is currently conducting, such as standing, sitting, walking, etc.), or to facilitate the diagnostics process for doctors or physicians by automatically categorizing the result (i.e. sensor data) based on the patterns discovered and consequently giving suggestions (e.g., identifying various muscle activities under different circumstances from sEMG data and providing information indicating different muscle status such as muscle relaxation, muscle imbalance, etc.).
2. Supervised learning can be used to train the system. The function of this kind of algorithms is multi-facet. One application is training the system to provide personalized posture control. For example, consider a user (assuming that he is a student) going to school on a daily basis. When he is sitting in a classroom having a class, he can set the alert to be vibration only and adopt a setting of sensitivity at a higher level than that when conducting other activities such as walking. After a few learning attempts, the system will “remember” the settings for the specific occasion—sitting in the classroom—in this example. Likewise, during other occasions such as walking, taking a bus or doing exercise, the different settings will be applied based on the learning of the user's previous setting for each occasion. This is achieved by using the supervised machine-learning algorithm, utilizing various kinds of context information such as sensor data, GPS, time etc. as inputs for identifying the “occasion” and the user's setting as the output to conduct the training process.
3. Another important functionality that is delivered through supervised machine learning is to provide automatic analysis and diagnosis based on sensor readings. One can train the system using sensor readings with corresponding expert opinions (diagnosis, instruction etc.). The system can learn and remember the diagnosis and instructions made by the experts 262 (doctors, specialists) previously for each type of sensor readings. When the same condition happens (as identified by the sensor readings), the system will try to provide diagnosis and instructions based on the knowledge it has learned. This automatic diagnostic function can be used for providing the patient with more meaningful results (by providing diagnostic results for different sensor readings). It can also provide doctors/physicians a diagnostic reference that is acquired from the machine learning process based on previous diagnosis for similar conditions from other experts.

### A.8. Adaptive UI 242

Adaptive UI 242 provides a conceptual layer that hides the platform dependencies from the application logic, enabling the decoupling of user-interface design and application design. With this concept, different type interfaces can be offered to the user based on the rules of the user, and also based on the type of the user access device 240 the user is using. For example, when the user is using an IOS device as the user access device 240, the results (feedback) will be provided via an IOS notification system, and also provided to the built-in HealthKit for deep integration with the mobile devices. When the user switches to an android device, the feedback will be adapted to an android OS, utilizing available infrastructures (e.g., Google Fit) on that OS. When a doctor or a specialist accesses his or her patient's data via a web interface, the adaptive UI 242 will be switched to a different view that is tailored for the doctor or the specialists, e.g., showing statistics, previous instructions, progression of the symptom, etc.

### A.9. Open API 244

To further enhance extensibility of the proposed system, an Open API 244 is also developed to provide third-party developers 264 most essential features from the system. The extensibility is delivered by three means, namely, extensions, third-party apps, and wrappers for existing service infrastructures. Extensions can be built to further enrich the functionality of the system; it becomes a part of the infrastructure once added. In addition, full-fledged third-party apps can be built using provided API. The wrapper is a way to bridge the system to the existing service infrastructures such as social network services (Facebook, Twitter), as well as professional healthcare platforms (myoclinic.org).

The openness and extensibility is crucial for building a healthy ecosystem surrounding the system. Via this ecosystem, patients, doctors and developers can be connected together, forming a large community.

By connecting to existing social network services and healthcare platforms, the social connections are also utilized to achieve an effective and efficient social-telemedicine approach at a global scale. For example, the patient can find one who has a similar condition and exchange the information regarding diagnosis and treatment, while doctors can also collaborate with each other in the same manner, giving patients diagnosis, instruction and suggestions.
A.10. Advantages of the System Over Existing Ones

The system as disclosed above has several advantages over existing ones.
Conventional brace is bulky and uncomfortable. The system is realized in a form a garment, offering comfort to the patient.
Many existing diagnostic approaches are conducted in hospital/laboratory environment. A doctor or a specialist cannot acquire long-term real-time diagnostic surveillance data from the patient. On the other hand, the system as disclosed above enables remote monitoring of the patient. The sensor data can be used by the doctor or the specialist to perform diagnosis.
In conventional approaches, data analysis and intervention techniques can only be provided in a hospital/laboratory environment. Diagnostic results cannot be delivered to the patients promptly whenever the patient needs them. The system disclosed above, on the other hand, enables prompt delivery of the results to the patient by sending the results to the user access device 240.
Existing posture control devices use simple feedback (normally audio/vibration alert), rather than providing more meaningful information to the users as feedback. The system disclosed above provides detailed personalized feedbacks.
Existing biofeedback devices use the threshold-based detection, lacking the ability to provide dynamic, personalized and adaptive feedbacks.
In the conventional biofeedback system design, the computation logic is mainly achieved on a microcontroller unit (MCU). It consumes a lot of power as the computation involved is complicated, so that the battery-support time of this conventional design is considerably short. Different from the conventional biofeedback system design, the system as disclosed above generates the personalized feedbacks in the computational space 204 but not in the wearable space 202, allowing the electrical power in the garment to be solely used for the sensors 222, 224, the sensor interface 220 and the SCU 210, and thereby lengthening the battery-support time provided by the garment.

### B. The Present Invention

Generalizing the exemplary design of the sensor-based wearable biofeedback system disclosed above yields the present invention that is detailed as follows.

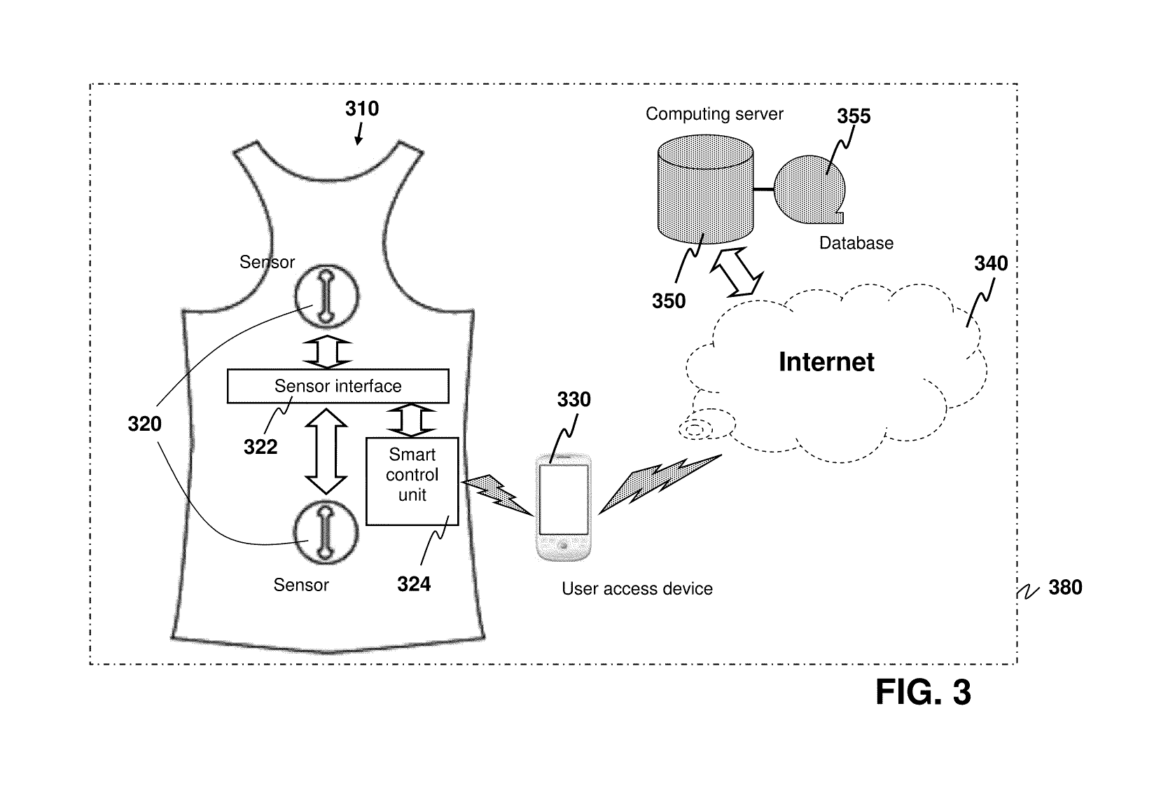

FIG. 3 depicts a garment and a biofeedback means in accordance with an exemplary embodiment of the present invention. A garment 310, which is used for monitoring patient-related signals of a patient having scoliosis and thereby enabling the patient to obtain a personalized biofeedback based on the patient-related signals, comprises plural sensors 320, a sensor interface 322 and a SCU 324. The patient-related signals are signals measured by sensors 320 installed in the garment 310, and are useful to indicate different states of the patient, such as positions at different parts of his or her body, in order that useful information can be extracted for use in providing the personalized biofeedback as medical intervention against the scoliosis that the patient suffers.

In particular, the sensors 320, the sensor interface 322 and the SCU 324 are integrated in the garment 310. It allows the patient-related signals to be non-intrusively measured by the sensors while maintaining comfort to the patient when the patient wears the garment. The SCU 324 is communicable with the sensors 320 via the sensor interface 322, and is configured to aggregate the patient-related signals measured by the sensors. Furthermore, the SCU 324 is configured to be communicable with a computing server 350 outside the garment 310 via a user access device 330. The function of the computing server 350 is as follows. The computing server 350 is configured to process the aggregated patient-related signals sent from the SCU 324 to generate the personalized biofeedback, and configured to forward the personalized feedback to the user access device 330 for presentation to the patient. For the garment 310, it is configured to electrically power the sensors 320, the sensor interface 322, the SCU 324 but neither the user access device 330 nor the computing server 350. An important advantage is that the personalized biofeedback is obtainable by the patient without a need for the garment 310 to spend electrical power to process the patient-related signals in generating the personalized biofeedback. Powering the sensors 320, the sensor interface 322 and the SCU 324 by the garment 310 is achievable by, e.g., having one or more batteries installed in the garment 310.

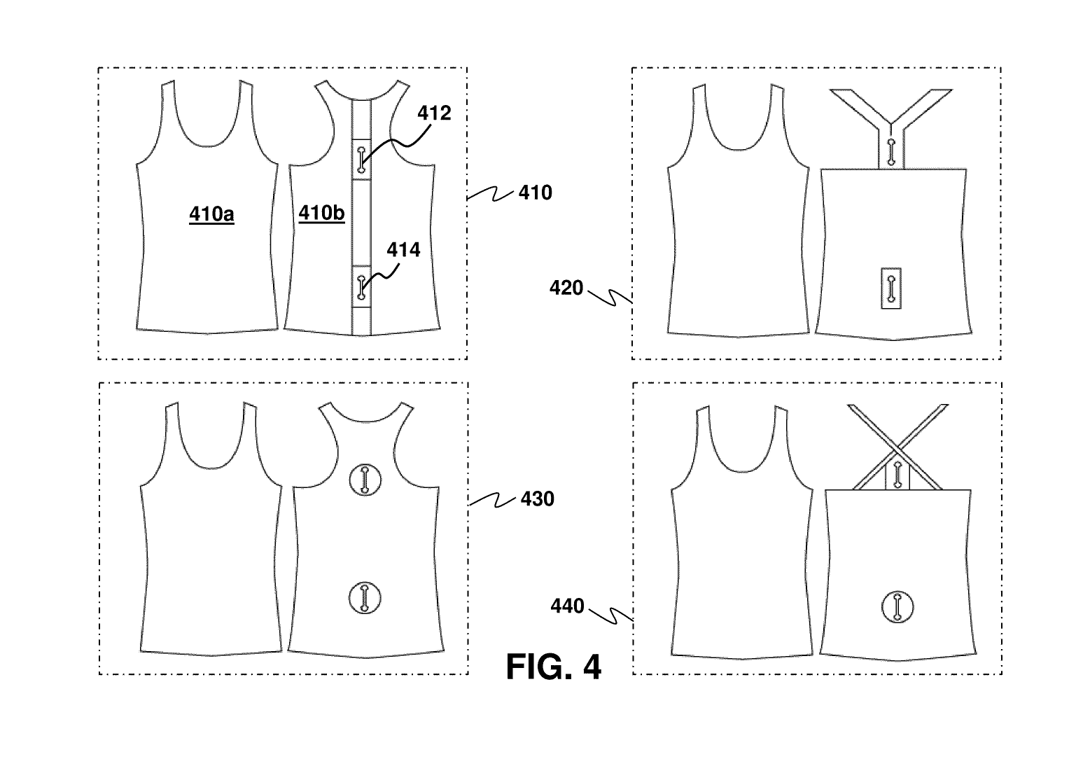

Since the sensors 320 are mostly located around the backbone of the patient as the patient suffers from scoliosis, preferably the garment 310 is fabricated as a tank top, i.e. a shirt without sleeves. FIG. 4 shows four examples of such tank top (410, 420, 430, 440) equipped with sensors. As the four tank tops 410, 420, 430, 440 are similar, the tank top 410 is used here as an example for illustration. The tank top 410 has a front side 410a and a back side 410b. Two sensors 412, 414 are installed in the tank top 410. The first sensor 412, installed on the neck portion of the back side 410b, is used to measure the position or coordinate of the patient at his or her neck. The second sensor 414 is located on the waist portion of the back side 410b and is used for measuring the position or coordinate of the patient's backbone around the waist.

Preferably, the sensors 320 comprise one or more physical sensors and one or more virtual sensors. As is mentioned above, an individual virtual sensor comprises a plurality of component sensors such that plural data measured by the component sensors in one measurement are processed to from a single patient-related data of said individual virtual sensor. Examples of physical sensors include a 3-axis accelerometer, a 3-axis gyroscope, a magnetometer, a surface electromyography sensor, a temperature sensor, and a humidity sensor. An example of virtual sensors is a compliance detector.

It is also preferable that the sensor interface 322 is configured to support one or more communication protocols for communicating with the SCU 324 and the sensors 320, where the one or more protocols are selected from i²c, serial communication and WBAN.

A system 380 for monitoring patient-related signals of a patient having scoliosis and for providing a personalized biofeedback to the patient based on the patient-related signals is realizable by including the garment 310, the user access device 330 and the computing server 350.

Preferably in the system 380, the computing server 350 is configured to execute one or more machine learning algorithms in processing the aggregated patient-related signals. The one or more machine learning algorithms may include an unsupervised-learning algorithm and/or a supervised learning algorithm. The unsupervised-learning algorithm may be used for: identifying the patient' behavior; discovering one or more patterns from the aggregated patient-related signals to automatically categorizing a result based thereon so as to facilitate a diagnostic process; or providing information indicating different muscle status. The function performed by the supervised-learning algorithm can be: training the computing server 350 to provide personalized posture control; or training the computing server 350 to provide automatic analysis and diagnosis from the patient-related signals measured by the sensors 320.

Because of many advantages as mentioned above, the computing server 350 is preferably a cloud-based server. The computing server 350 is connectable to the user access device 330 via, e.g., the Internet 340.

The computing server 350 may be further configured to store a copy of the aggregated patient-related signals in a database 355. The database 355 may be a cloud-based database.

The user access device 330 may be a mobile-computing device such as a smartphone or a tablet. Typically, the user access device 330 is accompanied with the patient while the computing server 350 is located remotely away from the patient.

In one option, the SCU 324 and the user access device 330 are configured to communicate with each other by Bluetooth 4.0 LE. The user access device 330 may be configured to use a software framework. In one option, the software framework has an interface to interface with a cloud infrastructure, and handles communications from the SCU. In addition, the software framework may provide required libraries and interfaces for one or more mobile platforms such as iOS or Android. The software framework may also provide an API.

Optionally, the system is further adapted for treating AIS.

The present invention may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

What is claimed is:

1. A garment for monitoring patient-related signals of a patient having scoliosis and thereby enabling the patient to obtain a personalized biofeedback based on the patient-related signals, the garment comprising plural sensors, a sensor interface and a smart control unit (SCU), wherein:
the plural sensors, the sensor interface and the SCU are integrated in the garment, allowing the patient-related signals to be non-intrusively measured by the sensors while maintaining comfort to the patient when the patient wears the garment;
the SCU is communicable with the sensors via the sensor interface and is configured to aggregate the patient-related signals measured by the sensors;
the SCU is configured to be communicable with a computing server outside the garment via a user access device, where the computing server is configured to process the aggregated patient-related signals sent from the SCU to generate the personalized biofeedback, and configured to forward the personalized feedback to the user access device for presentation to the patient; and
the garment is configured to electrically power the sensors, the sensor interface and the SCU but neither the user access device nor the computing server so that the personalized biofeedback is obtainable by the patient without a need for the garment to spend electrical power to process the patient-related signals in generating the personalized biofeedback.

2. The garment of claim 1, wherein the garment is fabricated as a tank top.

3. The garment of claim 1, wherein the sensors comprise one or more physical sensors and one or more virtual sensors, an individual virtual sensor comprising a plurality of component sensors such that plural data measured by the component sensors in one measurement are processed to from a single patient-related data of said individual virtual sensor.

4. The garment of claim 3, wherein the one or more physical sensors are selected from one or more of a 3-axis accelerometer, a 3-axis gyroscope, a magnetometer, a surface electromyography sensor, a temperature sensor and a humidity sensor.

5. The garment of claim 3, wherein the one or more virtual sensors include a compliance detector.

6. The garment of claim 1, wherein the sensor interface is configured to support one or more communication protocols for communicating with the SCU and the sensors, the one or more protocols being selected from i²c, serial communication and WBAN.

7. A system for monitoring patient-related signals of a patient having scoliosis and for providing a personalized biofeedback to the patient based on the patient-related signals, the system comprising:
the garment of claim 1;
a user access device configured to communicate with the SCU for at least receiving the aggregated patient-related signals; and
a computing server configured to communicate with the user access device, to process the aggregated patient-related signals received from the user access device so as to generate the personalized biofeedback, and to forward the personalized biofeedback to the user access device for presentation to the patient.

8. The system of claim 7, wherein the SCU and the user access device are configured to communicate with each other by Bluetooth 4.0 LE.

9. The system of claim 7, wherein the user access device is a mobile-computing device.

10. The system of claim 9, wherein the mobile-computing device is a smartphone or a tablet.

11. The system of claim 7, wherein the user access device is configured to use a software framework.

12. The system of claim 11, wherein the software framework has an interface to interface with a cloud infrastructure, and handles communications from the SCU.

13. The system of claim 11, wherein the software framework provides required libraries and interfaces for one or more mobile platforms.

14. The system of claim 13, wherein the one or more mobile platform include iOS or Android.

15. The system of claim 11, wherein the software framework provides an open API.

16. The system of claim 7, wherein the computing server is a cloud-based server.

17. The system of claim 7, wherein the computing server is further configured to store a copy of the aggregated patient-related signals in a database.

18. The system of claim 17, wherein the database is a cloud-based database.

19. The system of claim 7, wherein the computing server is configured to execute one or more machine learning algorithms in processing the aggregated patient-related signals.

20. The system of claim 19, wherein the one or more machine learning algorithms include an unsupervised-learning algorithm configured to perform a function selected from:
identifying the patient' behavior;
discovering one or more patterns from the aggregated patient-related signals to automatically categorizing a result based thereon so as to facilitate a diagnostic process; and
providing information indicating different muscle status.

21. The system of claim 19, wherein the one or more machine learning algorithms include a supervised learning algorithm configured to perform a function selected from:
training the computing server to provide personalized posture control; and
training the computing server to provide automatic analysis and diagnosis from the patient-related signals measured by the sensors.

22. The system of claim 19, wherein the system is adapted for treating adolescent idiopathic scoliosis.
